(19) United States
(12) Patent Application Publication
(10) Pub. No.: US 2024/0042364 A1
(43) Pub. Date: Feb. 8, 2024

(54) AIR PURIFIER WITH ROTATABLE FILTER

(71) Applicant: BISSELL Inc., Grand Rapids, MI (US)

(72) Inventors: Derek Smith, Greenville, MI (US); Mackenzie Knapp, Grand Rapids, MI (US); Brian Dexter, Grand Rapids, MI (US)

(21) Appl. No.: 18/228,120

(22) Filed: Jul. 31, 2023

Related U.S. Application Data
(60) Provisional application No. 63/394,402, filed on Aug. 2, 2022.

(51) Int. Cl. B01D 46/00; B01D 46/69; B01D 46/26; A61L 9/20

(52) U.S. Cl. CPC B01D 46/0056; B01D 46/0049; B01D 46/0028; B01D 46/69; B01D 46/26; A61L 9/20; B01D 2265/06; B01D 2279/65; B01D 2273/30; A61L 2209/12; A61L 2209/14

(57) ABSTRACT

An air purifier and rotatable filter assembly for inside air. The air purifier includes a housing. The rotatable filter assembly is disposed in the housing. A filter assembly support is disposed in the housing. The rotatable filter assembly is coupled to the filter assembly support. The filter assembly support supports the rotation of the rotatable filter assembly. The housing includes a filter access panel that is moveable to an access position. The filter access panel in the access position allows a user maintenance access to the rotatable filter assembly.

### BACKGROUND

Air purifying devices filter particles from air and/or kill airborne pathogens. Such devices typically use filters to purify the air surrounding the device. Yet there are continual issues in operation, function, and ornamental appearance that remain unresolved.

Some air purifiers utilize a cylindrical or multi-sided filter arrangement to increase airflow and filter area. While these configurations provide some benefits, the user experience accessing the filter area for replacement is less than ideal. For example, in order to replace such an air filter, it is often necessary to remove the top or bottom of the air purifier and flip the unit upside down. Other air purifiers utilize a drawer system to provide access to the air filter area, but force compromises in other areas because of the physical interference they create — for example with lighting and other internal elements.

An air purifier with a filter replacement system that enables more convenient removal and replacement of filters that also avoids substantial interference with light elements and other components inside the filter chamber is needed.

### BRIEF DESCRIPTION

An air purifier with improved maintenance access to a rotatable filter is provided herein. Aspects of the disclosure relate to an air purifier including a housing, a rotatable filter, a filter support, and a blower. The rotatable filter is configured to rotate relative to the filter support to provide improved maintenance access to the rotatable filter assembly. The rotatable filter can be referred to as a rotatable filter assembly and the filter support can be referred to as a filter support assembly.

The air purifier housing includes an air inlet, an air outlet, and an air treatment chamber. The air inlet, air treatment chamber, and air outlet are fluidly connected by an air flow path. The blower may be disposed within the housing and configured to draw air into the housing through the air inlet along the airflow path through the rotatable filter, and push air out of the housing through the air outlet.

The rotatable filter assembly can be configured to at least one of filter, clean, and purify air flowing through the air flow path during operation. The rotatable filter assembly may include a plurality of replaceable air treatment panels. The rotatable filter assembly can include a rotatable filter assembly frame configured to selectively receive the plurality of replaceable air treatment panels. The replaceable air treatment panels selectively and removably couple to the rotatable filter assembly frame to facilitate easy removal while the rotatable filter assembly is installed within the air treatment chamber of the air purifier.

The filter assembly support is configured to support the rotation of the rotatable filter assembly relative to the rotatable filter assembly. Different replaceable air treatment panels of the rotatable filter assembly can be selectively accessible within the housing for removal as the rotatable filter assembly rotates relative to the filter assembly support. In this way, the panels can be replaced with new panels or cleaned and reseated in the rotatable filter assembly without removing the rotatable filter assembly frame from the housing.

The housing may include a selectively removable filter access panel moveable between a closed position and an access position. The selectively removable filter access panel can provide selective access to the rotatable filter assembly installed within the housing.

Some embodiments of the air purifier may include a light element within the air treatment chamber. The light element can be installed within the air treatment chamber such that the rotatable filter assembly can rotate relative to the filter support without interfering with the light element. Selective removal of an air treatment panel from the rotatable filter assembly can facilitate installation or removal of a light element installed in the air treatment chamber.

These and other features and advantages of the present disclosure will become apparent from the following description of particular embodiments, when viewed in accordance with the accompanying drawings and appended claims.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. In addition, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components. Any reference to claim elements as “at least one of X, Y and Z” is meant to include any one of X, Y or Z individually, and any combination of X, Y and Z, for example, X, Y, Z; X, Y; X, Z; and Y, Z.

### BRIEF DESCRIPTION OF THE DRAWINGS

In the drawings:

FIG. 1 is a perspective view of an air purifier with a rotatable filter assembly according to various aspects described herein;

FIG. 2 is a front view of the air purifier of FIG. 1 with a filter access panel in an access position;

FIG. 3 is a cross-sectional view through the line III-III of the air purifier of FIG. 1 with the filter access panel in an access position;

FIG. 4 is a front view of the air purifier of FIG. 2 with a replaceable air treatment panel removed;

FIG. 5 is a perspective view of the air purifier of FIG. 2, with a replaceable air treatment panel exploded from a rotatable filter assembly of the air purifier;

FIG. 6 is a cross-sectional view through the line VI-VI of the air purifier of FIG. 1 with the filter access panel in an access position;

FIG. 7 is a perspective view of a plurality of replacement air treatment panels packaged for transportation according to one aspect of the disclosure;

FIG. 8 is a close-up side view of a connection between a replaceable air treatment panel and a filter assembly frame according to one aspect of the disclosure;

FIG. 9 is an expanded view of section IX of the air purifier of FIG. 3;

FIG. 10 is a modified sectional of an air purifier according to one aspect of the disclosure;

FIG. 11 is an expanded view of section XI of the air purifier of FIG. 10;

FIG. 12 is a modified sectional of an air purifier according to one aspect of the disclosure;

FIG. 13 is an expanded view of section XIII of the air purifier of FIG. 12;

FIG. 14 is a modified sectional of an air purifier according to one aspect of the disclosure;

FIG. 15A is an expanded view of section XV of the air purifier of FIG. 14;

FIG. 15B is an expanded view of section XV of the air purifier of FIG. 14 when a rotatable filter assembly is not in an access position;

FIG. 16 is a perspective view of an air purifier with a rotatable filter assembly according to one aspect;

### DETAILED DESCRIPTION

Aspects of the disclosure relate to an air purifier and/or a rotatable filter assembly. While primarily discussed herein in terms of an air purifier for indoor air, aspects of the rotatable filter assembly and embodiments thereof disclosed herein are applicable to other types of air filtration apparatuses.

The term “clean” as used herein is to describe the removal of pollutants from air as compared to the ambient air. Pollutants can include dirt, dust, volatile organic compounds (VOCs), biological contaminants (e.g., bacteria, viruses, mold spores, waste products, etc.), soot particles, and any other pollutants that can be found in indoor and/or outdoor flows.

The term “purify” is used herein to describe killing or inactivating biological contaminants, pathogens, or microorganisms in the ambient air. It is noted that the air purifier apparatus can have a variety of applications including commercial or home based application.

As used herein, the term “upstream” refers to a direction that is opposite the air flow direction, and the term “downstream” refers to a direction that is in the same direction as the air flow. Additionally, as used herein, the terms “radial” or “radially” refer to a direction away from a common center. Furthermore, as used herein, the term “set” or a “set” of elements can be any number of elements, including only one.

All directional references (e.g., radial, axial, proximal, distal, upper, lower, upward, downward, left, right, lateral, front, back, top, bottom, above, below, vertical, horizontal, clockwise, counterclockwise, upstream, downstream, etc.) are only used for identification purposes to aid the reader's understanding of the present disclosure, and do not create limitations, particularly as to the position, orientation, or use of aspects of the disclosure described herein. Connection references (e.g., attached, coupled, secured, fastened, connected, and joined) are to be construed broadly and can include intermediate members between a collection of elements and relative movement between elements unless otherwise indicated. As such, connection references do not necessarily infer that two elements are directly connected and in fixed relation to one another. The exemplary drawings are for purposes of illustration only and the dimensions, positions, order, and relative sizes reflected in the drawings attached hereto can vary.

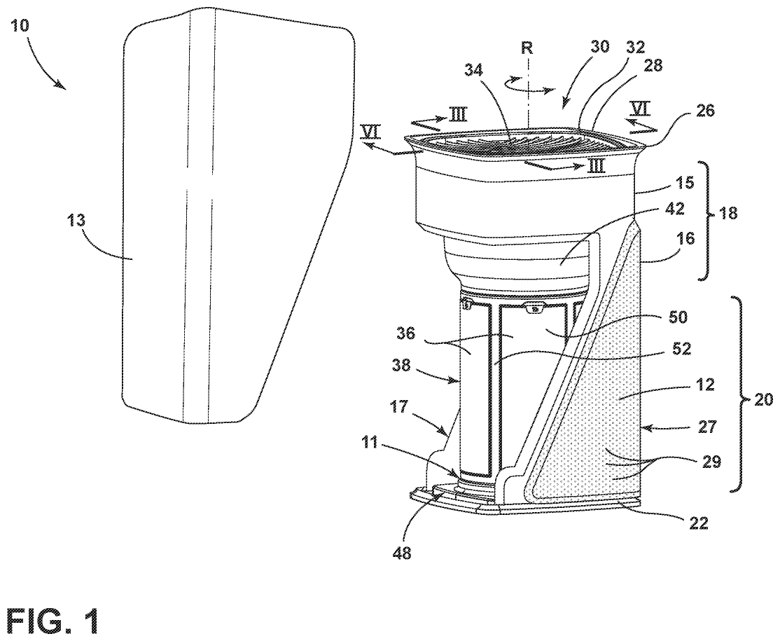

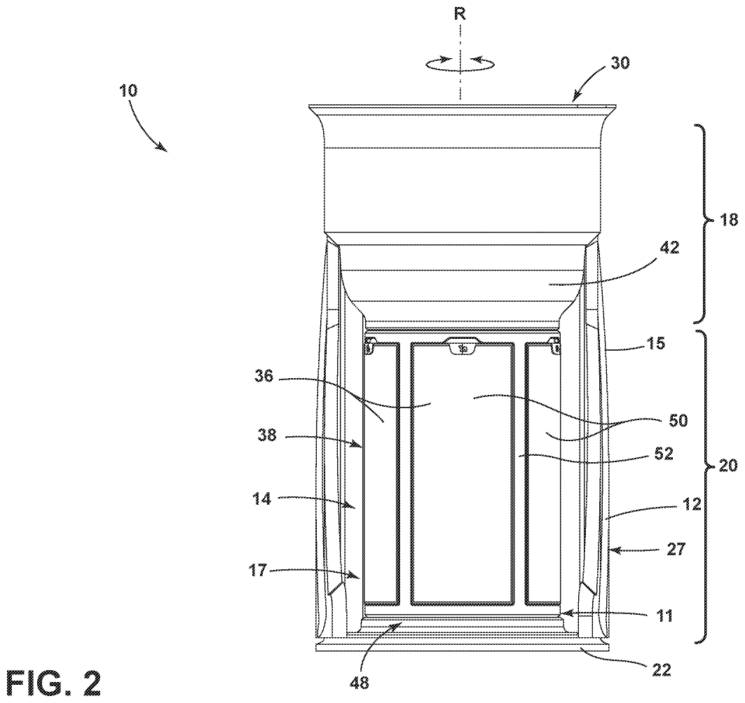

FIGS. 1-6 illustrate an exemplary air purifier 10 including a housing 12. The housing 12 may include a filter access panel 13 removably coupled to a primary structure 15. The air purifier 10 includes an air inlet 27, an air outlet 30, and an air treatment chamber 25. In one embodiment, the housing 12 defines the air inlet 27, air outlet 30, air treatment chamber 25, and filter access panel 13. The air inlet 27, air treatment chamber 25, and air outlet 30 are fluidly connected by an air flow path. A rotatable filter system 11 may be disposed within the housing 12. The rotatable filter system 11 can include a rotatable filter assembly 38 and a filter assembly support 48. The filter assembly support 48 can be coupled to the rotatable filter assembly 38. A blower system may be disposed within the housing 12. As shown in FIG. 2, an interior 14 of the air purifier 10 can at least partially be defined by the housing 12. The rotatable filter assembly 38 and blower system are disposed within the interior 14 of the air purifier 10.

The housing 12 can be a monolithic body, e.g., a single body that is a single, non-separable piece, or formed as a single unitary piece at manufacture, as opposed to being formed by combining separate elements into one during manufacture. Alternatively, the housing 12 can be defined by more than one component coupled together. The shape of the housing 12, as illustrated by way of example is a prism, having a base or cross-sectional shape that is a squircle (e.g., a shape intermediate between a square and a circle), however it is contemplated that the housing 12 can be any shape, such as, but not limited to, a cylinder having a base or cross-sectional shape that is a circle, a round rectangular prism having a base or cross-sectional shape that is a rounded rectangle or a square, or an elliptic cylinder having a cross-sectional shape that is an ellipse or oval.

As shown in FIG. 1, the housing 12 defines an exterior periphery 16 for the air purifier 10. The housing 12 can further define an upper portion 18 and a lower portion 20. The upper portion 18 and the lower portion 20 can divide the housing 12 in half vertically. However, it is contemplated that the upper portion 18 can be greater than or less than half of the housing 12, with the lower portion 20 defining the remaining portion of the housing 12. The lower portion 20 can include a base 22. Optionally, the base 22 can include legs or other supporting structures.

The air inlets 27 can be provided in various locations and configurations on the housing 12. By way of example, the air inlets 27 can include a plurality of perforations 29 in the lower portion 20 of the housing 12. The plurality of perforations 29 can be included on one or any number of sides of the housing 12. In the current embodiment three sides of the housing 12 have a set of perforations 29 that each permit air to enter into the air treatment chamber 25. The plurality of perforations 29 can be generally uniform or dynamic in size, number, and shape. In the depicted embodiment, the perforations 29 are organized into a generally rectangular pattern on one side of the housing 12 and a generally triangular pattern on two sides of the housing 12. In some embodiments, the filter access panel 13 can include perforations that act as air inlets while the filter access panel is disposed in a closed position.

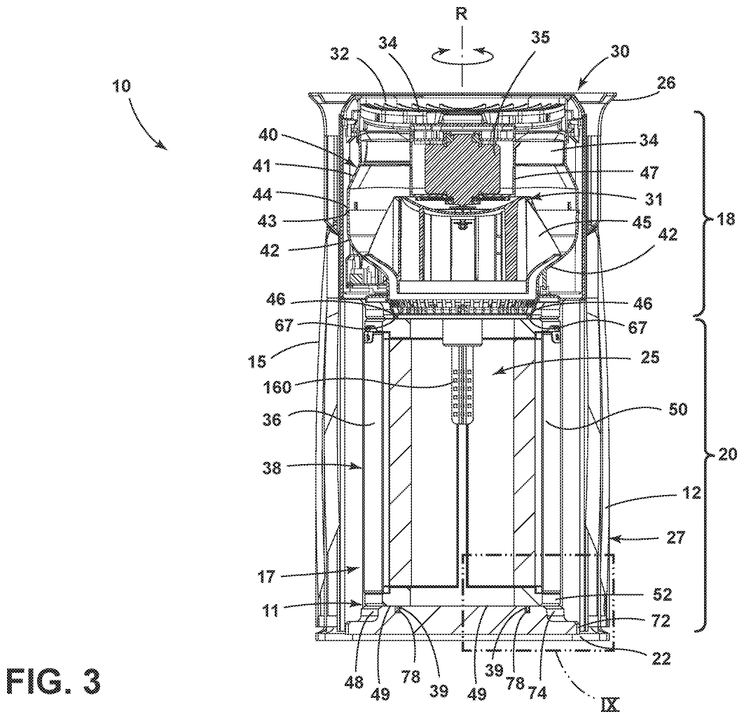

Referring to the sectional view of FIG. 3, an exemplary blower system 31 is illustrated. The blower system 31 is configured to move air from the air inlet 27 through the rotatable filter assembly 38 to the air outlet 30. By way of non-limiting example, the blower system 31 can include a set of fan blades 45 coupled to a fan motor 35 to drive the fan blades 45 in order to generate airflow, which is channeled toward the air outlet 30 by a fan shroud 40. In general, while the fan motor 35 is energized, air is drawn through the air inlet 27, the air treatment chamber 25, the fan shroud 40, and pushed out of the air outlet 30.

The illustrated embodiment of the fan shroud 40 has a two-piece construction with a lower fan shroud 42 and an upper fan shroud 41. The top end of the lower fan shroud 42 includes a channel 43 that receives the lower edge 44 of the upper fan shroud 41 to form the complete fan shroud 40. In alternative embodiments the fan shroud 40 can have a monolithic construction, e.g., a single body that is a single, non-separable piece, or formed as a single unitary piece at manufacture, as opposed to being formed by combining separate elements into one during manufacture. The shape of the fan shroud 40, as illustrated by way of example is generally vase-shaped where the bottom surface of an airflow grill 33 of the lower shroud 42 is generally flat, the shroud body curves outward to facilitate airflow around the motor housing 47, then back inward to facilitate airflow through openings between flow diverters 34 of the upper fan shroud 41 and out the air outlet 30. In alternative embodiments, the fan shroud 40 can have a different shape that suitably facilitates airflow from the air treatment chamber 25 to the air outlet 30. Optionally, as depicted in FIG. 3, the lower fan shroud 42 may define an upper rotation channel 46. In one embodiment, one or more motion aids 76 can be located in the upper rotation channel 46. The upper rotation channel 46 can help support the rotation of the rotatable filter assembly 38.

In one embodiment, the blower system 31 may be disposed in the upper portion 18 of the housing 12, and the rotatable filter assembly 38 may be disposed in the lower portion 20 of the housing 12, generally below the blower system 31. The blower 31 and the rotatable filter assembly 38 are separated by the airflow grill 33 of the lower fan shroud 42. That is, in the current embodiment, the airflow grill 33 of the lower fan shroud 42 generally separates the rotatable filter assembly 38 in the air treatment chamber 25 in the bottom portion 20 of the housing 12 from the fan shroud 40 and blower 31 in the upper portion 18 of the housing 12. In the current embodiment, the rotatable filter assembly 38 is disposed within the airflow path upstream of the blower 31 such that air is drawn from the air inlet 27 through the rotatable air treatment assembly 38. The blower system 31 is generally disposed downstream of the rotatable filter assembly 38 within the air flow path, regardless of the spatial arrangement of the components. However, other spatial arrangements are possible, for example where the blower 31 pushes air through the air treatment components, or a combination of pushing air through one or more air treatment components and drawing air through one or more other air treatment components.

Returning to FIG. 1, the filter access panel 13 can provide access to the rotatable filter assembly 38 for maintenance. The filter access panel 13 is moveable from a closed position to an access position. As depicted in FIG. 1, the filter access panel 13 is in the access position revealing a filter access aperture 17. In the depicted embodiment, the closed position of the filter access panel 13 has the filter access panel 13 is coupled to the primary structure 15 and the filter access panel 13 is in the access position when the filter access panel 13 is uncoupled from the primary structure 15. In an alternate embodiment, the filter access panel 13 may be hinged or otherwise movably coupled to the primary structure 15. In another embodiment, the filter access panel 13 and the primary structure 15 can be formed as one integral component. In one embodiment, the filter access panel 13 can be made from a breathable material. In one embodiment, the filter access panel 13 can include a plurality of air inlets 27.

A germicidal light source 160 is shown in one embodiment in FIG. 3. The germicidal light source 160 can be configured to treat the air in the airflow path as the air passes proximal to the germicidal light source 160. Alternatively or in addition, in some embodiments, the germicidal light source 160 can be configured to activate or work in concert with filter media installed in the rotatable filter. In one embodiment, the germicidal light source 160 is an ultraviolet (UV) light bulb, specifically a UV-C bulb. The germicidal light source 160 may be disposed within the housing 12. More specifically, the germicidal light source 160 may be disposed in the air treatment chamber 25. In one embodiment, the germicidal light source 160 can be selectively coupled to the lower fan shroud 42. In the depicted embodiment, the germicidal light source 160 is coupled to the airflow grill 33. As depicted, the germicidal light source 160 includes a plurality of germicidal light fasteners 161 that snap around a center ring 162 of the airflow grill 33. In alternative embodiments, the germicidal light source 160 can be mounted elsewhere within the air treatment chamber or mount to other structure or components of the air purifier 10. The germicidal light source 160 can be removably mounted in the air purifier 10 such that it can be easily removed from the housing and replaced as needed. In one embodiment, the germicidal light source 160 may be removed through the filter access aperture 17 when the filter access panel 13 is in the access position. Some embodiments may not include a germicidal light source 160 at all.

As shown in FIG. 1, a top portion 26 of the housing 12 is illustrated as being provided adjacent the upper portion 18. A lip or ledge 28 can define a perimeter of the top portion 26. The ledge 28 can be uniformly formed with the housing 12 or be a separate component of the housing 12. In the illustrated example, and by way of non-limiting example the top portion 26 has a similar shape to a cross-section of the housing 12. That is, the top portion 26 is generally shaped as a squircle, although this need not be the case. That is, the housing 12 can have a cross-section similar in shape to the top portion 26, alternatively, the top portion 26 can have a different shape or be a different size than a cross-section of the housing 12. The top portion 26 can surround the outside upper edge of the housing 12 to provide a clean aesthetic edge. In the current embodiment, the top portion 26 is joined to the upper portion 18 of the housing 12 and an upper fan shroud top edge 32.

The air outlet 30 can be provided in various locations and configurations on the housing 12. For example, the air outlet 30 can be provided toward the top end of the air purifier housing 12. In the current embodiment, the air outlet 30 is disposed within the internal perimeter of the top portion 26 of the housing 12, and may be referred to as a vent. The vent can include a plurality of flow diverters 34, which define a plurality of openings therebetween. While illustrated generally as radially extending vanes, the flow diverters 34 can be uniform or dynamic in essentially any shape, size, number, profile, spacing, layering, and orientation to provide a desired venting profile and aesthetic appearance. The flow diverters 34 can be single piece components or assemblies defined by two or more flow diversion components.

A user interface can be integrated or disposed on the housing or provided on a remote device in communication with the air purifier 10. For example, a user interface can be provided integrated into the top portion 26 of the housing 12. More specifically, the user interface can be located at an upper surface of the air purifier 10, although this need not be the case and the user interface can be provided at essentially any suitable location. It is contemplated that the user interface can be located on one or more sides of the housing 12. In a non-limiting example, the user interface can be operably coupled to the housing 12. This can include, but is not limited to, being mounted to or formed with the housing 12. It is further contemplated that the air purifier 10 can include more than one user interface. Optionally, the air purifier 10 can be remotely controlled by one or more electronic devices such as, but not limited to, a handheld remote or a cellular phone. The user interface can include or be coupled to a controller. The user interface can include various features including, but not limited to, indicator lights providing information about the air purifier 10 to the user and/or buttons that adjust one or more settings or monitor the status of one or more components of the air purifier 10.

The air purifier 10 can be powered by an external power supply, such as a household power supply, or by an internal power supply, such as a battery pack.

Perhaps as best shown in FIG. 2, the rotatable filter assembly 38 can include a filter assembly frame 52 configured to receive a plurality of replaceable air treatment panels 36. The replaceable air treatment panels 36 may alternately be referred to as facets. The depicted rotatable filter assembly 38 is a rotatable filter carousel. The term “carousel” as used herein refers to a rotating machine or device. As depicted, the rotatable filter assembly 38 is a modular filter assembly. In one embodiment, the rotatable filter assembly 38 is a pre-filter or includes a pre-filter. The rotatable filter assembly 38 can be configured to filter air flowing through the air flow path. The term “filter” as used herein as a verb encompasses cleaning air, purifying air, disinfecting air, and otherwise removing contaminants from air.

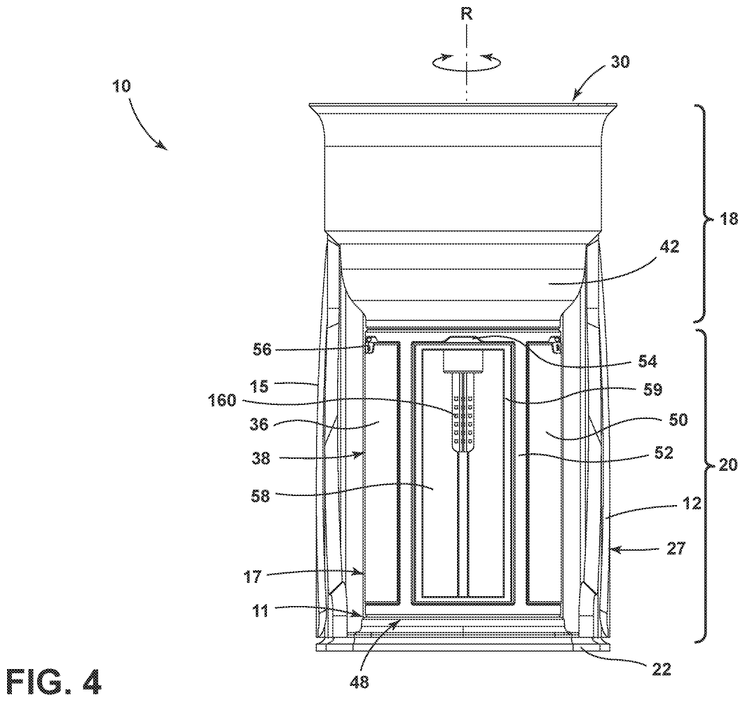

FIG. 4 shows a front view of the air purifier 10 with the replaceable air treatment panel 36 in the front and accessible position removed according to one embodiment. As depicted, the filter assembly frame 52 is cylindrical and has a circular base or cross-sectional shape that differs from the squircle base or cross-sectional shape of the housing 12. However, it is contemplated that the rotatable filter assembly 38 can be essentially any suitable shape, including shapes that substantially match that of the housing 12, such that the rotatable filter assembly 38 is harmonious with the exterior periphery 16 of the purifier 10.

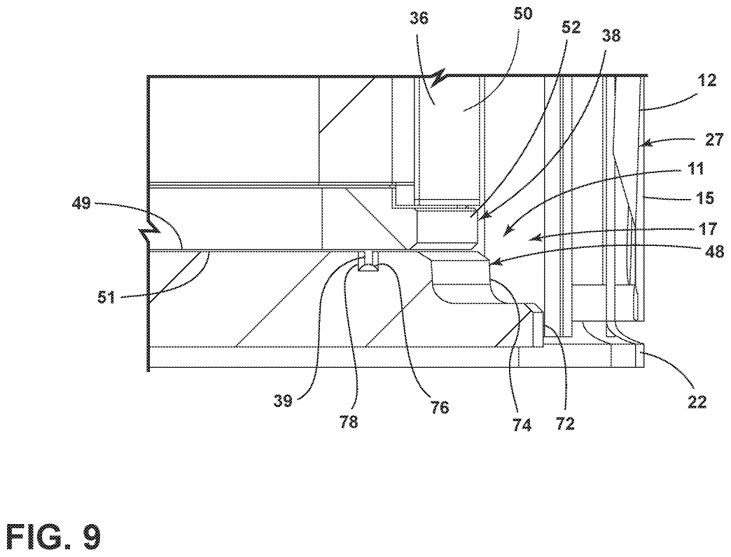

As shown in FIGS. 3 and 9, a lower coupling or alignment protrusion 39 can extend from a bottom surface 49 of the rotatable filter assembly 38 at a distance offset from the outer perimeter of the rotatable filter assembly 38. The lower coupling protrusion 39 may rotatably couple the rotatable filter assembly 38 to the filter assembly support 48. In one embodiment, the lower coupling protrusion 39 may extend from the bottom of the filter assembly frame 52 in a circular pattern offset from the perimeter of the filter assembly frame 52. An upper coupling or alignment protrusion 67 can extend from a top surface of the filter assembly frame 52 in a circular pattern offset from the perimeter of the filter assembly frame 52. The upper coupling protrusion 67 can rotatably couple the rotatable filter assembly 38 to the lower fan shroud 42 through the upper rotation channel 46. The coupling between the upper coupling protrusion 67 and the lower fan shroud 42 may support or assist the rotation of the rotatable filter assembly 38. In one embodiment, the upper coupling protrusion 67 may extend from the top surface of the filter assembly frame 52 on the perimeter of the filter assembly frame 52. While the FIG. 3 embodiment includes alignment protrusions 39, 67 on the bottom and top portions of the rotatable filter assembly 38, respectively, that cooperate to facilitate rotation of the rotatable filter assembly. In some alternative embodiments, one of the alignment protrusions can be foregone and the rotatable filter assembly 38 can still rotate with respect to the housing 12 and filter support 48. For example, the materials of the bottom surface 49 of the rotatable filter assembly 38 and an upper surface 51 of the filter assembly support 48 can be selected to have a relatively low coefficient of friction with respect to each other such that the rotatable filter assembly 38 can move relative to the filter assembly support 48 without any aid. The alignment protrusions 39, 67 may be used to align the rotatable filter assembly 38 and the filter assembly support 48 without permitting the rotation.

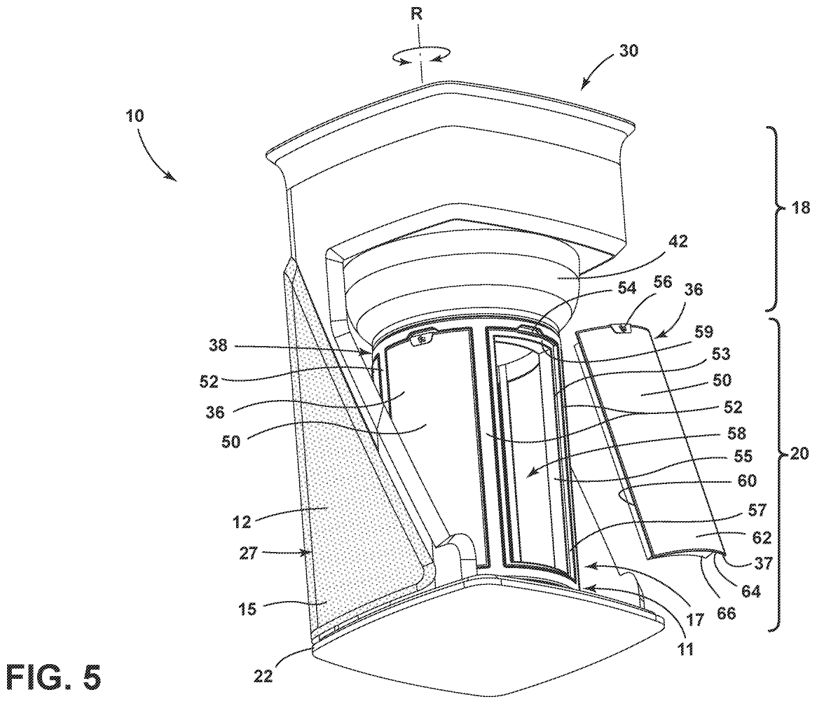

Referring to FIG. 5, the filter assembly frame 52 includes five air treatment panel seats 58 and five channels 54. Each air treatment panel seat 58 generally includes a perimeter surface 57 of the filter assembly frame 52 that defines an air treatment panel receiving aperture 53 as well as a panel support ledge 59 that extends from the perimeter surface 57 of the frame 52 further defining the air treatment panel receiving aperture 53. A panel support ledge inner surface 55 extends from the panel support ledge 59. Each perimeter surface 57, panel support ledge 59, panel support ledge inner surface 55, and air treatment panel receiving aperture 53 cooperatively form one of the air treatment panel seats 58 configured to receive and align one of the air treatment panels 36. Alternative constructions can include additional or fewer air treatment panel seats 58 and panels 36. Each channel 54 can be adjacent one of the air treatment panel seats 58. As depicted, the channel 54 is a channel with a seat.

The replaceable air treatment panels 36 can include a panel frame 37 and a filter media The filter media 50 may be contained by the panel frame 37. In one embodiment, the filter media 50 can be coupled to the panel frame 37. The replaceable air treatment panel 36 can include a panel coupler 56. In one embodiment, the replaceable air treatment panels 36 may be pre-filters. The replaceable air treatment panels 36 may be plastic components manufactured via molding or other suitable method. As depicted, the panel frame 37 is slightly curved to maintain the generally cylindrical shape of the filter assembly frame 52. However, in alternative embodiments the panel frame 37 can be a different shape. The filter media 50 can be shaped to compliment and interfit with the shape of the panel 37. Alternatively, the filter media 50 can be a rectangular cuboid or another shape and conform to the shape of the panel frame upon installation in the panel frame.

The panel frame 37 may include a perimeter surface 60 that defines a filter media portion 62. The filter media portion 62 can house the filter media 50. In one embodiment, a filter media mesh (not shown) can extend across the filter media portion 62 to retain the filter media 50 in the filter media portion 62. A frame coupling ledge 64 may extend along an underside of the perimeter surface 60. A retention ledge 66 can extend from the frame coupling ledge 64. In the depicted embodiment, as the replaceable air treatment panel 36 is seated in the air treatment panel seat 58, the frame coupling ledge 64 abuts the panel support ledge 59 and the retention ledge 66 abuts the panel support ledge inner surface 55.

The filter media 50 can substantially fill the filter media portion 62. Alternatively, the filter media 50 can fill less than all of the filter media portion 62, for example by gathering at the bottom of the panel frame 37, leaving some open space unfilled by the filter media 50.

The filter media 50 can be configured to remove odors from the air being exhausted from the air purifier 10 via the air outlet 30, and may capture, absorb, and/or remove odor causing VOCs. In certain embodiments, the filter media 50 is chosen from carbon, zeolite (understood to include at least zeolite X, zeolite Y, ultrastable zeolite Y, ZSM zeolite, offretite, and beta zeolite, among others), silica gel, faujasite, chabazite, clinoptilolite, mordenite, silicalite, metal organic frameworks, metal oxide, polymers, resins, and combinations thereof. In one embodiment, the filter media 50 is activated carbon pellets mixed with zeolites specific to ammonia removal.

In one embodiment, the rotatable filter assembly 38 includes at least one air channel that allows air exiting the air purifier 10 to bypass the filter media 50 so as to not suffocate the air purifier 10.

As depicted in FIG. 3, the rotatable filter assembly 38 can at least partially surround the germicidal light source 160. In one embodiment, the rotatable filter assembly 38 can fully surround the germicidal light source 160. The rotatable filter assembly 38 rotates around a rotation axis R, which, in the depicted embodiment, substantially aligns with the germicidal light source 160. The rotatable filter assembly 38 does not interfere with the germicidal light source 160 because the rotatable filter assembly 38 rotates around the germicidal light source 160 and does not contact it. Additionally, the rotatable filter assembly 38 may be spaced a sufficient distance from the germicidal light source 160 to allow unrestricted airflow through the rotatable filter assembly 38 to the airflow grill 33.

The germicidal light source 160 can have a photocatalytic oxidation (PCO) reaction with the filter media 50 in the air treatment panels 36 to help purify the air. For example, the UV-C light can react with a catalyst in the filter media 50 to convert malignant contaminants into water, carbon dioxide, and detritus. The detritus builds up in the filter media 50 and when the filter media 50 is saturated the replaceable air treatment panel 36 can be replaced. As depicted, the replaceable air treatment panels 36 are oriented to be parallel to the germicidal light source 160. The vertical orientation of the replaceable air treatment panels 36 creates a fixed distance between the filter media 50 and the germicidal light source 160, thereby allowing the replaceable air treatment panel 36 to be treated substantially evenly by the germicidal light source 160.

In one embodiment, the rotatable filter assembly 38 may have the shape of a conical frustum such that the replaceable air treatment panels 36 are at a non-parallel angle with respect to the germicidal light source 160. The angle of the replaceable air treatment panels 36 allows all of the filter media 50 to have a PCO relationship with the germicidal light source 160 because the germicidal light source 160 does not reach the bottom of the rotatable filter assembly 38 and the portion of the replaceable air treatment panels 36 that are further away from the germicidal light source 160 in the y-direction may be tilted inward so they are closer to the germicidal light source 160 in the x-direction. In one embodiment, the cylindrical rotatable filter assembly 38 may be less expensive to manufacture than the conical rotatable filter assembly 38.

The rotatable filter assembly 38 can be configured to contain any amount and/or type of filter media 50 required for a specific odor rich area. For example, the rotatable filter assembly 38 can contain activated carbon pellets used to adsorb VOCs and mixed with Zeolites specific to ammonia removal to more effectively remove cat litter odors from a litter box area. Also, a user can customize the filter media 50 inside the replaceable air treatment panels 36 used in the rotatable filter assembly 38 depending on the specific odor to address. Users can benefit from both the effective odor removal and the customizability of the filter media 50 to meet their odor removal requirements.

As noted above, in some embodiments, the replaceable air treatment panels 36 can be replenished with a new filter media 50. Alternatively, the replaceable air treatment panels 36 can be configured as a disposable component, with the replaceable air treatment panels 36 being disposed of when saturated and replaced with a new replaceable air treatment panel 36. Where the replaceable air treatment panels 36 or a portion thereof is configured for disposal after use, recyclable, biodegradable and/or compostable material, or combinations thereof, may be used to manufacture the replaceable air treatment panels 36.

According to another aspect of the disclosure, a replaceable air treatment panel 36 can include a one piece panel frame having a filter media matrix. The panel frame may be a plastic component manufactured via injection molding, e.g. with the matrix integrally formed by the molding process. The matrix can have a honeycomb configuration. After injection molding, a filter media is added to the matrix, for example by filling the matrix with a granulate, particulate, or pelletized filter media. Mesh can be disposed on the upper and lower sides of the panel frame to cover the matrix and contain the filter media within the matrix.

As shown in FIG. 5, the panel coupler 56 may removably join to the channel 54 to secure the replaceable air treatment panel 36 to the filter assembly frame 52. This allows the replaceable air treatment panels 36 to be modular and to be individually removed from the rotatable filter assembly 38 as needed, for example when the filter media 50 is fully saturated. The replaceable air treatment panels 36 may be attached to the filter assembly frame 52 by any suitable means, for example the air treatment panels 36 may be snapped or screwed to the filter assembly frame 52.

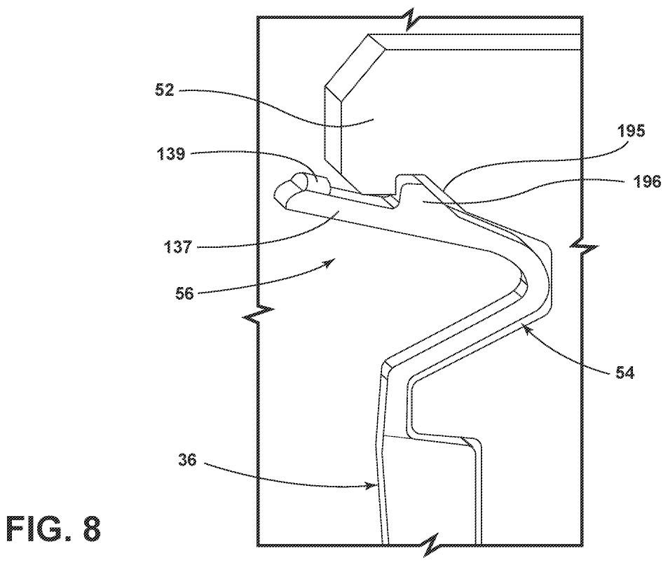

FIG. 8 illustrates a close up partial sectional view of an engaged snap fit joint between a panel coupler 56 of a replaceable air treatment panel 36 and filter assembly frame 52 according to one aspect. In this embodiment, installation of a replaceable air treatment panel 36 into the filter assembly frame 52 begins with the pressing portion 139 at the top of the panel 36 being depressed to cause the bias arm 137 to deflect for insertion of the snap fit panel coupler 56 into the channel 54 on the filter assembly frame 52 with the panel coupler protrusion 196 being unimpeded by the channel 54 on the filter assembly frame 52. The pressing portion 139 can then be released such that the bias arm 137 expands to contact a wall of the channel 54 with the protrusion 196 of the panel-side coupler 56 interfitting with and being secured by the frame-side catch 195 of the filter assembly frame 52.

Pressure may be applied to the pressing portion 139 to release the protrusion 196 of the snap fit panel coupler 56 from the catch 195 of the channel 54 and allow the replaceable air treatment panel 36 to be removed from the rotatable filter assembly 38. The removable coupling between the panel coupler 56 and the channel 54 allows the replaceable air treatment panels 36 to be individually and selectively removed from the rotatable filter assembly 38 as desired, for example when the filter media 50 is fully saturated or otherwise near end of life. By virtue of having discrete air treatment panels 36 that are individually replaceable, the rotatable filter assembly 38 provides modularity and simplifies filter replacement.

The modularity of the replaceable air treatment panel 36 improves the user's ability to replace the interior filter (in this case, the rotatable filter assembly 38). In one embodiment, the replaceable air treatment panels 36 may be fixedly coupled to the filter assembly frame 52 and the rotatable filter assembly 38 may be removed as needed. In one embodiment, all of the replaceable air treatment panels 36 are the same size and all of the air treatment panel seats 58 are the same size. In this way, the installation and removal processes for the replaceable air treatment panels 36 are simplified because each replaceable air treatment panel 36 fits in each air treatment panel seat 58.

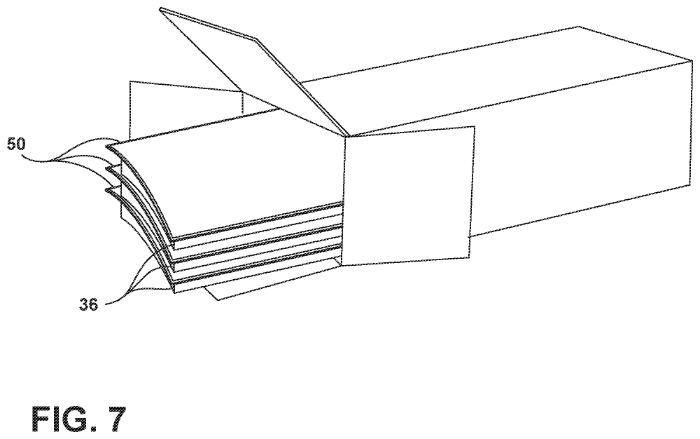

The replaceable air treatment panels 36 may be easier to produce than larger filter arrangements. The replaceable air treatment panels 36 can be easier to scale and use in other air purifiers 10 and other products with larger or smaller filter areas than the air purifier 10. The replaceable air treatment panels 36 may be easier to ship in more compact arrangements than other filter arrangements. For example, as depicted in FIG. 7, many of the replaceable air treatment panels 36 may be stacked together for transportation. In contrast, circular filters that cannot be collapsed take up more room in containers for transportation, thereby making them more difficult and/or more expensive to transport.

In one embodiment, two or more different configurations of the replaceable air treatment panel 36 may be used in the rotatable filter assembly 38. For example, the rotatable filter assembly 38 can include some air treatment panels 36 with the filter media 50 coupled to the panel frame 37 and some air treatment panels 36 with a filter media matrix. In one embodiment, air treatment panels 36 containing different filter media 50 can be used in the rotatable filter assembly 38.

As depicted in FIG. 3, the filter assembly support 48 is rotatably coupled to the rotatable filter assembly 38. The filter assembly support 48 is configured to support the rotation of the rotatable filter assembly 38 about the rotation axis R. As depicted, the filter assembly support 48 is stationary. In one embodiment, the filter assembly support 48 can be the base 22 of the air purifier 10 or another component configured to support the rotation of the rotatable filter assembly 38. In one embodiment, the filter assembly support 48 can be a platform. The platform may include a base platform portion 72 and an upper platform portion 74. In an alternate embodiment, the platform can be one monolithic piece. As depicted, the upper platform portion 74 has a smaller diameter than the base platform portion 72. In an alternate embodiment, the upper platform portion 74 may have the same diameter as the base platform portion 72 or a larger diameter than the base platform portion 72. As depicted, both the base platform portion 72 and the upper platform portion 74 are generally cylindrical. In another embodiment, the base platform portion 72 and the upper platform portion 74 may be any other suitable shape. In one embodiment, the base platform portion 72 and the upper platform portion 74 can have different shapes. In one embodiment, the base platform portion 72 and the upper platform portion 74 may generally conform to the shape of the housing 12. The base platform portion 72 and the upper platform portion 74 may be secured together by any suitable joining method such as adhesion, gluing, bonding, welding, or the like. In one embodiment, the base platform portion 72 can be secured to the base 22 of the housing 12.

As shown in FIG. 9, the upper platform portion 74 of the filter support 48 can define a rotation channel 78 offset from the perimeter of the upper platform portion 74. In one embodiment, the rotation channel 78 may be sized and shaped to correspond to the lower coupling protrusion 39 of the rotatable filter assembly 38. One or more bearings or motion aids 76 may be placed in the rotation channel 42 to assist the rotation of the rotatable filter assembly 38. In one embodiment, the rotation channel 78 may not include any motion aids 76. As depicted, the motion aids 76 are friction reducing hemispheres that are stationary within the rotation channel 78. In another embodiment, the motion aids 76 may move within the rotation channel 78.

The rotatable filter assembly 38 is moveably coupled to the filter assembly support 48 through the rotation channel 78. As depicted, the lower coupling protrusion 39 of the rotatable filter assembly 38 extends into the rotation channel 78 to contact the friction reducing hemisphere 76. While the lower coupling protrusion 39 is engaged with the friction reducing hemispheres 76, the bottom surface 49 of the rotatable filter assembly 38 can be configured not to engage with the upper surface 51 of the filter assembly support 48. Put another way, the lower coupling protrusion 39 can be configured such that interfacing with motion aid 76 maintains a gap or an amount of space between the bottom surface 49 of the rotatable filter assembly 38 and the upper platform portion 74 of the filter assembly support 48 to facilitate lower friction rotation. The friction reducing hemispheres 76 generally have a lower coefficient of friction than the rotation channel 78 and thus allow the rotatable filter assembly 38 to rotate more easily when a user applies a rotation force. A user can rotate the rotatable filter assembly 38 to access each of the replaceable air treatment panels 36 while the filter assembly support 48 (and housing 12) remains stationary.

As shown in FIG. 1, the rotation of the rotatable filter assembly 38 can provide selective maintenance access to the replaceable air treatment panels 36 as the rotatable filter assembly 38 rotates about the rotation axis R with the filter access panel 13 in the access position. The term “maintenance access” as used herein refers to any and all access to the rotatable filter assembly 38, including, but not limited to, removing the replaceable air treatment panels 36, replacing the removable air treatment panels 36, and cleaning either the replaceable air treatment panels 36 or the rotatable filter assembly 38 while it remains installed in the air purifier 10. In one embodiment, for example, the rotatable filter assembly 38 may be vacuumed to remove debris rather than be removed and replaced with a new rotatable filter assembly 38 or new replaceable air treatment panels 36.

With the filter access panel 13 is in the access position, a user can rotate the rotatable filter assembly 38 to provide selective maintenance access to the replaceable air treatment panels 36 through the filter access aperture 17. Put another way, the replaceable air treatment panels 36 can be accessed through the filter access aperture 17 in the housing 12 when the filter access panel 13 is in the access position, and the rotatable filter assembly 38 can rotate to vary which replaceable air treatment panel(s) 36 are accessible. This arrangement avoids interference with light elements inside of the air treatment chamber 25, for example, the germicidal light source 160 (perhaps as best shown in FIG. 3). In one embodiment, the rotatable filter assembly 38 may rotate such that a user has selective maintenance access to one of the replaceable air treatment panels 36 at a time. Having multiple replaceable air treatment panels 36 in the rotatable filter assembly 38 rather than one filter and permitting the rotatable filter assembly 38 to rotate allows the user to have maintenance access to the replaceable air treatment panels 36 from one location on the air purifier when the filter access panel 13 is in the access position without moving, tipping, or rotating the air purifier 10.

In general, the replacement air treatment panels 36 may be ready for maintenance at about the same time because they generally undergo the same amount of airflow. However, some replaceable air treatment panels 36 may be ready for maintenance before others for a variety of reasons. For example, the particular rotational positioning of the air treatment panels and particular airflow pattern at the inlets and through the purifier 10 can impact the saturation of the filter material and thus the replacement timing of the panels. The panels may include an end of life indicator to aid with replacement selection. The modularity of the replaceable air treatment panels 36 allows a user to only replace the air treatment panels 36 they desire to replace. In one embodiment, the rotatable filter assembly 38 can be rotated such that the air treatment panels 36 that are less saturated are relocated to different positions to improve the lifespan of the air treatment panels 36.

The rotatable filter and filter support of the rotatable filter system 11 can be configured to rotate a variety of different ways in various different embodiments. As discussed above in connection with FIGS. 1-6, a low friction bearing arrangement can be utilized to facilitate rotation. In some embodiments, different types of bearing arrangements can be utilized. For example, ball bearings, roller bearings, needle bearings, sleeve bearings, or other bearing relationships can be utilized to facilitate rotation between the filter and filter support.

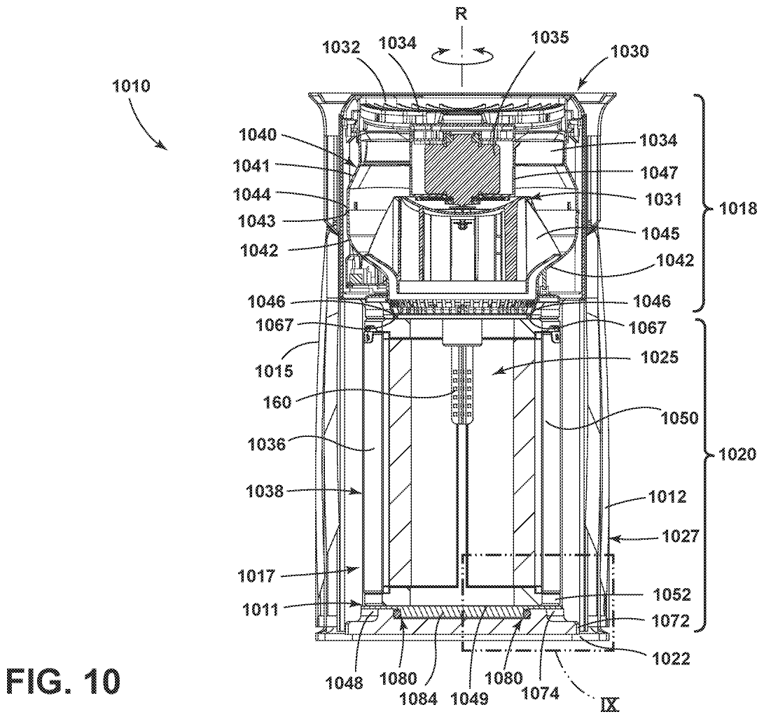

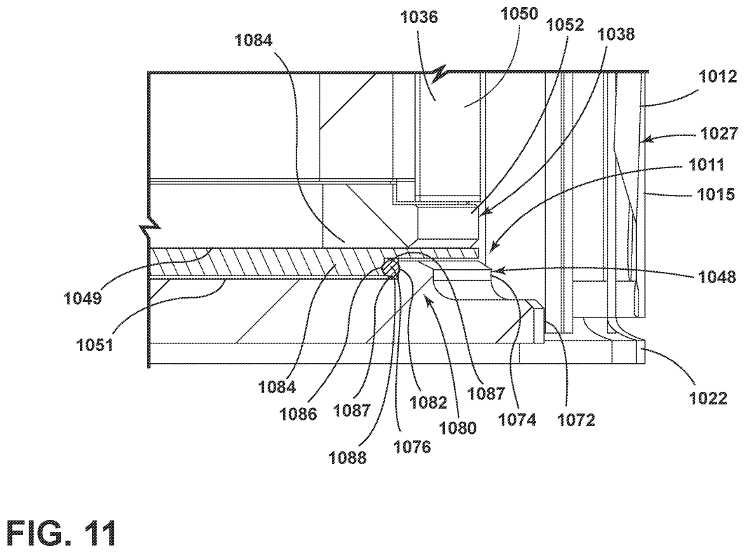

In one embodiment, the rotatable filter assembly 1038 may include a rotatable subfloor 1084 that carries the rotatable filter assembly frame 1052 such that rotation of the subfloor 1084 also causes the rotatable filter assembly frame 1052 along with its filter panels to rotate, as shown in described below with reference to FIGS. 10-11. The filter assembly frame 1052 can be selectively coupled to the rotatable subfloor 1084 to facilitate tandem rotation.

An alternative filter assembly support 1048 is depicted in FIG. 10. The air purifier 1010 has many of the same features as the air purifier 10 discussed with respect to the preceding figures except for the differences outlined below. Like elements are generally referred to with the same reference numeral with a leading “10.”

As depicted, a rotatable filter assembly 1038 is supported by and rotates relative to the filter assembly support 1048 through a bearing assembly 1080 (perhaps as best shown in the expanded view of section XI shown in FIG. 11).

The bearing assembly includes an inner raceway 1086, an outer raceway 1082, and a plurality of bearings 1076. The bearing assembly may also include a cage or separator 1087 for the bearings 1076.

In the current embodiment, the outer raceway 1082 is defined by the filter support 1048 and the inner raceway 1086 is defined by the rotatable filter assembly 1038. In alternative embodiments, the outer raceway may be defined by the rotatable filter assembly 1038 while the inner race may be defined by the filter support.

In the FIGS. 10-11 embodiment, the bearings or motion aids 1076 are rolling elements, and, more specifically, ball bearings. In another embodiment, a different motion aid or type of bearing may be used.

Together, the inner raceway 1086 of the rotatable filter assembly 1038 and the outer raceway 1082 of the filter support 1048 cooperatively define a bearing assembly channel 1088. The ball bearings 1076 are disposed between the inner and outer raceways 1086, 1082 to facilitate rotation of the filter assembly relative to the filter support. The inner and outer raceways 1086, 1082 can be substantially aligned to facilitate rotation via the ball bearings 1076.

In one embodiment, the bearing assembly 1080 includes three ball bearings 1076 equidistantly spaced by a separator or cage 1087. The ball bearings 1076 can be held in position relative to each other by the cage 1087 to permit the ball bearings 1076 to rotate within the channel while keeping them spaced a fixed distance apart. In another embodiment, the ball bearings 1076 may not be spaced equidistantly. In one embodiment, any suitable number of ball bearings 1076 may be included in the bearing assembly 1080 such that the load of the rotatable filter assembly 1038 is sufficiently supported permitting satisfactory rotation relative to the housing 1012.

In operation, as a user applies force to the rotatable filter assembly 1038, the rotatable subfloor 1084 and the inner raceway 1086 rotate relative to the outer raceway 1082 and cause the ball bearings 1076 to rotate as well. The rotating ball bearings 1076 have a lower coefficient of friction than if two flat surfaces were sliding against each other, and thus the ball bearings 1076 allow the rotatable filter assembly 1038 to rotate more easily. In one embodiment, the bearing assembly 1080 facilitates bidirectional rotation of the rotatable filter assembly 1038. In another embodiment, the bearing assembly 1080 may facilitate unidirectional rotation of the rotatable filter assembly 1038. In one embodiment, the rotatable subfloor 1084 may be removed and the filter assembly frame 1038 may define an inner raceway 1086 that rotates relative to the outer raceway 1082.

In one embodiment, a second bearing assembly may be included at the top of the rotatable filter assembly 1038. The second bearing assembly can operate in much the same way as the bearing assembly 1080 described above. The second bearing assembly may further assist the rotation of the rotatable filter assembly 1038 by easing some of the load on the bearing assembly 1080.

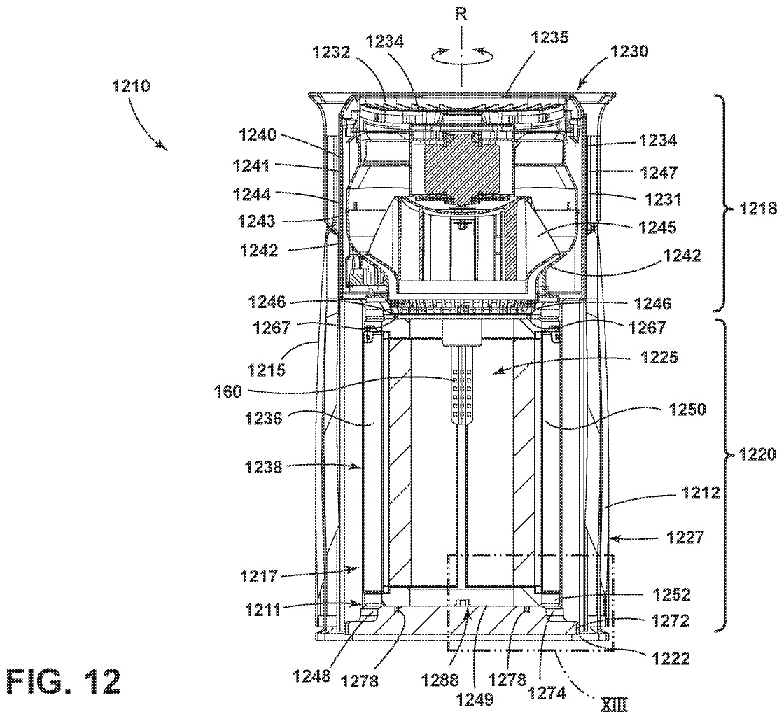

An alternative air purifier 1210 is depicted in FIG. 12. The air purifier 1210 has many of the same features as the air purifier 10 discussed with respect to FIGS. 1-6 except for the differences outlined below. Like elements are generally referred to with the same reference numeral with a leading “12.”

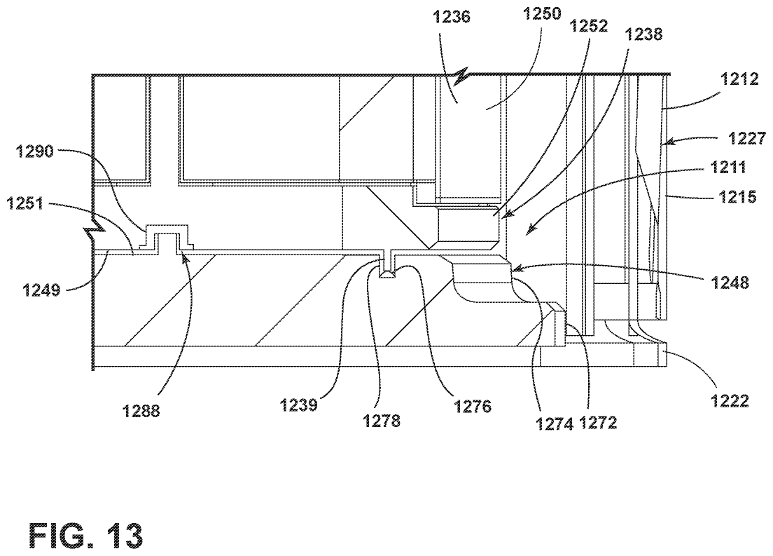

Perhaps as best seen in FIG. 13, an axle 1288 can extend from the upper platform portion 1274 of the filter assembly support 1248. In one embodiment, the axle 1288 is made from metal or reinforced plastic. The rotatable filter assembly 1238 may include a bushing 1290 in the bottom surface 1249 of the rotatable filter assembly 1238 configured to interface with the axle 1288. In another embodiment, the bushing 1290 is an indented portion of the bottom surface 1249. The bushing 1290 can couple to the axle 1288, and the axle 1288 creates a point of rotation for the rotatable filter assembly 1238 and allows the rotatable filter assembly 1238 to rotate more easily. As depicted, the filter assembly support 1248 includes a rotation channel 1278 including at least one motion aid 1276 that the lower coupling protrusion 1239 of the rotatable filter assembly 1238 contacts, and these elements can work in conjunction with the coupling between the axle 1288 and the bushing 1290 to ease the rotation of the rotatable filter assembly 1238. In another embodiment, the axle 1288 and the bushing 1290 may be used as the sole element to allow rotation of the rotatable filter assembly 1238. In yet another embodiment, the axle 1288 and the bushing 1290 may work in concert with other rotation enabling or assisting elements.

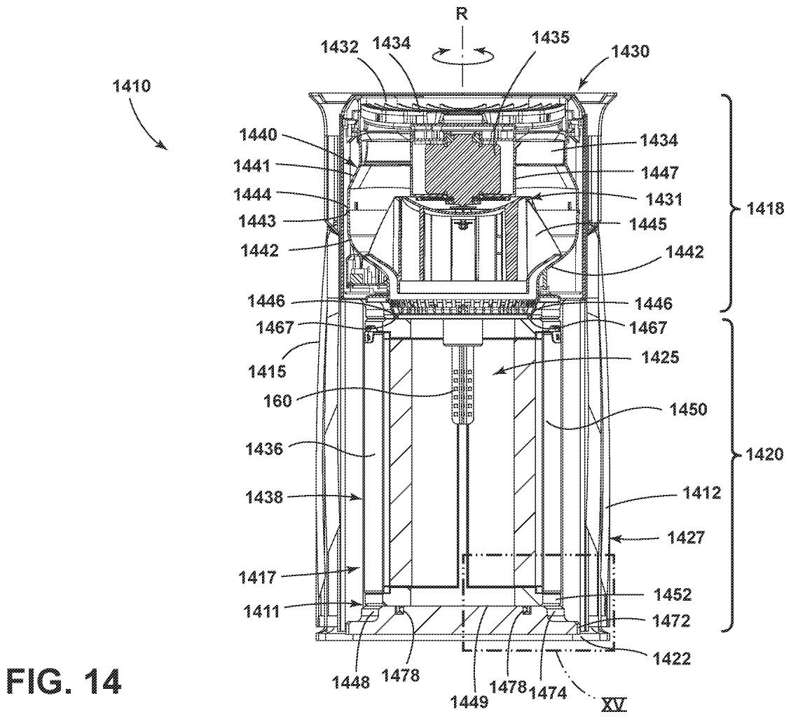

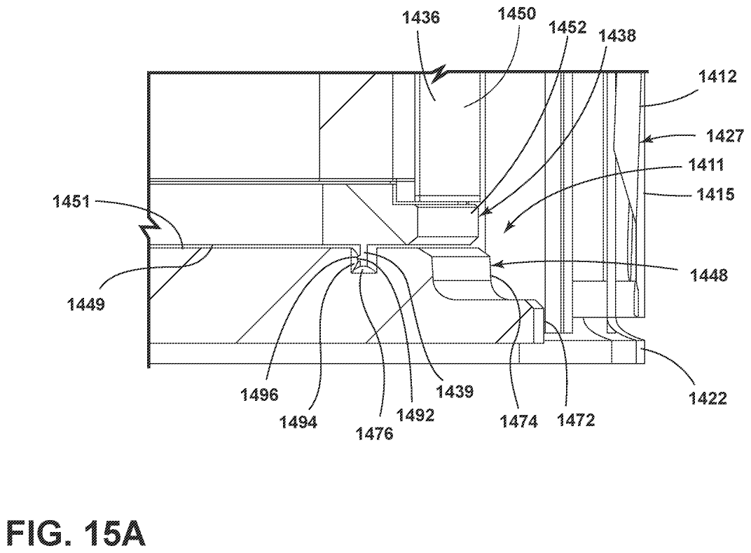

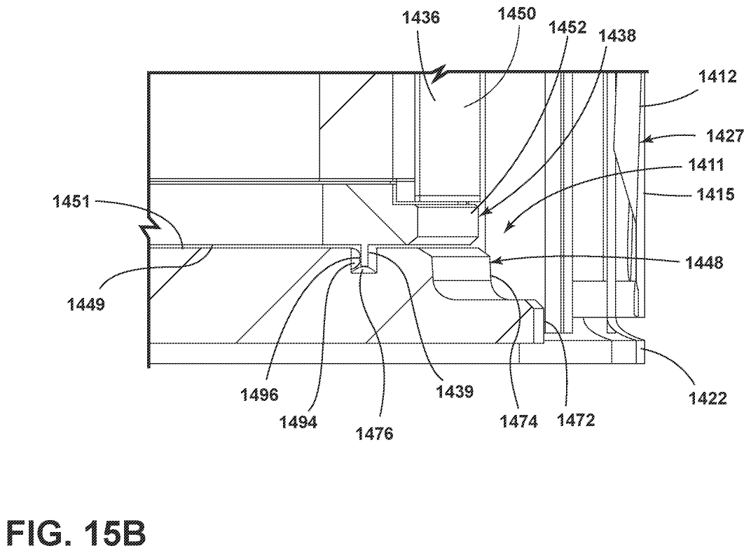

Optionally, the rotatable filter system can include a positioning system to indicate to a user when a replaceable air treatment panel is in a maintenance access position. FIG. 14 illustrates a rotatable filter system 1411 including a positioning system. In the depicted embodiment, the positioning system includes a detent 1494 extending from one side of the rotation channel 1478 and a catch 1492 extending from the corresponding surface on the lower coupling protrusion 1439 of the rotatable filter assembly 1438 (perhaps as best shown in FIG. 15A). The detent 1494 can define an opening 1496 sized and positioned to correspond to the catch 1492. In one embodiment, there are multiple detents 1494 spaced around the rotation channel 1478. In another embodiment, there may be one detent 1494 in the rotation channel 1478. For example, the sole detent 1494 can be placed in the rotation channel 1478 at the front of the air purifier 1410 to indicate when a replaceable air treatment panel 1436 is accessible through the filter access aperture 1417. In one embodiment, the lower coupling protrusion 1439 includes one catch for each of the replaceable air treatment panels 1436 in the rotatable filter assembly 1438.

As depicted in FIG. 15A, as the rotatable filter assembly 1438 rotates to a maintenance access position for one of the replaceable air treatment panels 1436, the catch 1492 may become lodged in the opening 1496. The contact between the catch 1492 and the detent 1494 can create some resistance that signals to a user that a replaceable air treatment panel 1436 is in position. In some embodiments, this may result in an audible click so that the user is audibly notified that a replaceable air treatment panel 1436 is in a maintenance access position. In some embodiments, the lower coupling protrusion 1439 may deflect away from the detent 1494 in response to contacting the detent 1494. The user may continue to rotate the rotatable filter assembly 1438 past the maintenance access position. In some embodiments, the user may have to apply a small amount of additional force to move the rotatable filter assembly 1438 past a maintenance access position. The position of the lower coupling protrusion 1439 and the detent 1494 when not in a maintenance access position is shown in FIG. 15B. As depicted, the rotatable filter assembly 1438 includes a motion aid 1476 in the rotation channel 1478. However, the positioning system described above can be used in embodiments that do not include the motion aid 1476.

In one embodiment, the detent 1494 may extend from the bottom of the rotation channel 1478. However, this can result in vertical motion of the rotatable filter assembly 1438 as the replaceable air treatment panels 1436 moved into and out of a maintenance access position. Vertical movement may affect the airflow through the air purifier 1410. Additional space can be added to the upper rotation channel 1446 (if present) to allow for vertical movement of the rotatable filter assembly 1438.

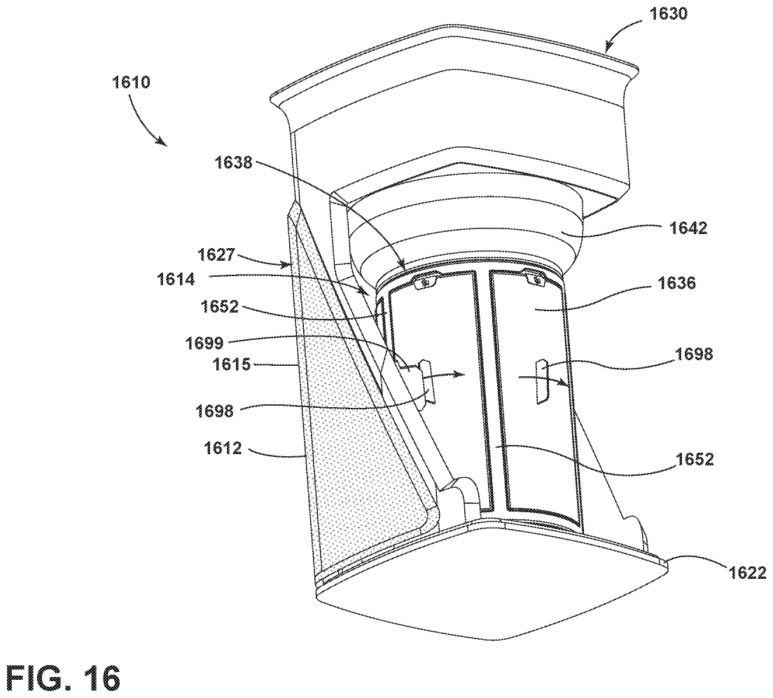

In one embodiment, the positioning system may use components of the air purifier other than the rotation channel. In FIG. 16, one such positioning means is shown. A tab 1698 can extend outward from the center of each replaceable air treatment panel 1636. A projection 1699 can extend from an inner surface of the housing 1612 into the interior 1614 of the housing 1612. As the rotatable filter assembly 1638 rotates, one of the tabs 1698 may contact the projection 1699 indicating that at least one of the replaceable air treatment panels 1636 is in a maintenance access position. The tabs 1698 can be configured to flex toward the replaceable air treatment panel 1636 as the tab 1698 passes the projection 1699. In one embodiment, the tabs 1698 may be manufactured from a flexible material. In one embodiment, the tabs 1698 can be moveably coupled to the replaceable air treatment panels 1636. The user may have to apply some additional force to move the tab 1698 past the projection 1699.

In one embodiment, the force of the tab 1698 moving past the projection 1699 may shake loose some debris from the replaceable air treatment panels 1636. In one embodiment, a vacuum with a crevice tool attachment or other suitable cleaning device can be used to remove the debris from the air purifier 1610. In one embodiment, a debris reservoir may be incorporated below the replaceable air treatment panels 1636 to capture the loose debris. The debris reservoir may be removeable for debris disposal or a cleaning device may be used to remove the debris from the debris reservoir.

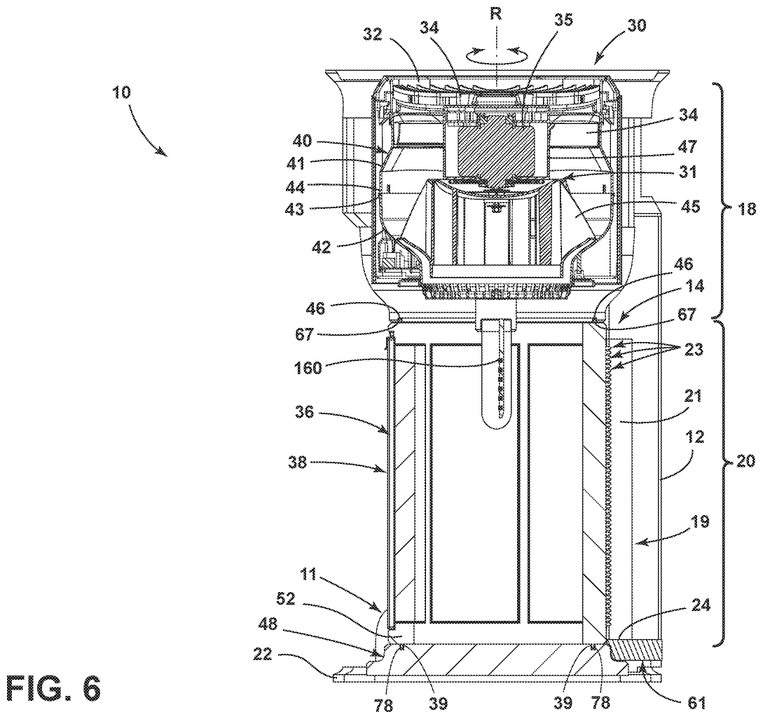

As depicted in FIG. 6, a filter cleaner may be disposed in the interior 14 of the housing 12 for cleaning the filter panels during rotation. In one embodiment, the filter cleaner is a brush 19 supported by the housing 12. The brush 19 can include a brush base 21 and a plurality of bristles 23 extending from the brush base 21. As depicted, the brush base 21 is secured to an interior surface of the housing 12. The plurality of bristles 23 may be configured to engage the plurality of replaceable air treatment panels 36 during rotation of the filter. The plurality of bristles 23 can dislodge particles from the air treatment panels 36 as the rotatable filter assembly 38 rotates. The dislodged particles can collect in a debris reservoir 24 below the brush.

In one embodiment, the debris reservoir 24 is selectively removable from the air purifier. That is, once a sufficient amount of particles (for example, debris or dust) have collected in the reservoir, the user can remove, empty, and return the reservoir. The debris reservoir 24 can be disposed toward the bottom of the interior 14 of the housing 12 such that as particles are dislodged from the filter media, they fall into the debris reservoir 24 located below the bristles 23. As depicted, the debris reservoir 24 can be slidably seated in a clean out channel 61. A user can pull the debris reservoir 24 out of the housing 12, the debris reservoir 24 may be emptied, and the debris reservoir 24 may be re-installed in the housing 12 through the clean out channel 61. In one embodiment, the debris reservoir 24 can be permanently installed in the housing 12 and may be emptied through a clean out port. For example, a vacuum may be applied to the clean out port to suck the particles out of the debris reservoir 24 or the air purifier 10 may be tilted to cause the particles to escape the debris reservoir 24 through the clean out port.

The terms “comprising” or “comprise” are used herein in their broadest sense to mean and encompass the notions of “including,” “include,” “consist(ing) essentially of,” and “consist(ing) of”. The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples.

The above description relates to general and specific embodiments of the disclosure. However, various alterations and changes can be made without departing from the spirit and broader aspects of the disclosure as defined in the appended claims, which are to be interpreted in accordance with the principles of patent law including the doctrine of equivalents. As such, this disclosure is presented for illustrative purposes and should not be interpreted as an exhaustive description of all embodiments of the disclosure or to limit the scope of the claims to the specific elements illustrated or described in connection with these embodiments. Any reference to elements in the singular, for example, using the articles “a,” “an,” “the,” or “said”, is not to be construed as limiting the element to the singular.

What is claimed is:

1. An air purifier comprising:
an air purifier housing defining an air inlet, an air treatment chamber, an air outlet, and a filter access panel;
a rotatable filter assembly disposed in the air treatment chamber, the rotatable filter assembly including a filter assembly frame configured to receive a plurality of replaceable air treatment panels, the rotatable filter assembly configured to rotate about a rotation axis;
a filter assembly support movably coupled to the rotatable filter assembly, the filter assembly support configured to support the rotation of the rotatable filter assembly; and
a blower system within the air purifier housing, the blower system configured to move air from the air inlet to the air outlet through the rotatable filter assembly,
wherein the filter access panel is moveable between a closed position and an access position,
wherein the filter access panel in the access position provides maintenance access to the rotatable filter assembly, and
wherein a different portion of the rotatable filter assembly is accessible as the rotatable filter assembly rotates.

2. The air purifier of claim 1, wherein the filter assembly support is a platform that the rotatable filter assembly sits atop.

3. The air purifier of claim 2, the filter assembly support further including a rotation channel, wherein the rotatable filter assembly movably couples to the filter assembly support through the rotation channel.

4. The air purifier of claim 3, the rotatable filter assembly further including a lower coupling protrusion extending from a bottom surface of the filter assembly frame, wherein the rotatable filter assembly movably couples to the filter assembly support through the lower coupling protrusion and the rotation channel.

5. The air purifier of claim 4, the filter assembly support further including at least one motion aid disposed in the rotation channel, the at least one motion aid being configured to ease the rotation of the rotatable filter assembly, wherein the lower coupling protrusion contacts the at least one motion aid.

6. The air purifier of claim 2, further comprising:
the filter assembly support further including an outer raceway, the outer raceway being configured to be stationary;
a rotatable subfloor coupled to the rotatable filter assembly, the rotatable subfloor including an inner raceway, the rotatable subfloor and the inner raceway configured to rotate, the inner raceway being substantially aligned with the outer raceway; and
at least one ball bearing disposed between the inner raceway and the outer raceway and in contact with the inner raceway and the outer raceway,
wherein the inner raceway and the at least one ball bearing rotate as the rotatable filter assembly is rotated.

7. The air purifier of claim 1, the filter assembly support further including:
a crossbar;
a rotation arm extending from the crossbar; and
a filter coupling protrusion extending from the rotation arm, the filter coupling protrusion configured to couple to the rotatable filter assembly.

8. The air purifier of claim 7, the filter assembly support further including a platform configured to support the rotatable filter assembly.

9. The air purifier of claim 1, wherein the plurality of replaceable air treatment panels are removably coupled to the filter assembly frame.

10. The air purifier of claim 1 further including:
a germicidal light source disposed within the air treatment chamber,
wherein the rotatable filter assembly is configured to at least partially surround the germicidal light source.

11. The air purifier of claim 1, wherein the rotatable filter assembly rotates to provide selective maintenance access to one of the plurality of replaceable air treatment panels at a time.

12. The air purifier of claim 1 further comprising:
a brush supported by the housing, the brush including a brush base and a plurality of bristles extending from the brush base, the bristles configured to contact the plurality of replaceable air treatment panels,
wherein the brush dislodges particles from the plurality of replaceable air treatment panels.

13. The air purifier of claim 12 further comprising a debris receptacle disposed below the bristles, wherein the particles dislodged from the plurality of replaceable air treatment panels by the brush are collected in the debris receptacle.

14. A rotatable filter system for use with an air purifier, the rotatable filter system comprising:
a rotatable filter assembly configured to rotate about a rotation axis, the rotatable filter assembly including:
a plurality of replaceable air treatment panels; and
a filter assembly frame configured to receive the plurality of replaceable air treatment panels; and
a filter assembly support configured to support the rotation of the rotatable filter assembly.

15. The rotatable filter system of claim 14, wherein the filter assembly support is a platform that the rotatable filter assembly sits atop.

16. The rotatable filter system of claim 15,
the filter assembly support further including a rotation channel; and
the rotatable filter assembly further including a lower coupling protrusion extending from a bottom surface of the filter assembly frame,
wherein the rotatable filter assembly movably couples to the filter assembly support through the lower coupling protrusion and the rotation channel.

17. The rotatable filter system of claim 16, the filter assembly support further including at least one motion aid disposed in the rotation channel, the at least one motion aid being configured to ease the rotation of the rotatable filter assembly, wherein the lower coupling protrusion contacts the at least one motion aid.

18. The rotatable filter system of claim 17, wherein the at least one motion aid is a friction reducing hemisphere.

19. The rotatable filter system of claim 15, further comprising:
the platform further including an outer raceway, the outer raceway being configured to be stationary;
a rotatable subfloor coupled to the rotatable filter assembly, the rotatable subfloor including an inner raceway, the rotatable subfloor and the inner raceway configured to rotate, the inner raceway being substantially aligned with the outer raceway; and
at least one ball bearing disposed between the inner raceway and the outer raceway and in contact with the inner raceway and the outer raceway,
wherein the inner raceway and the at least one ball bearing rotate as the rotatable filter assembly is rotated.

20. The rotatable filter system of claim 16,
the rotation channel including a detent extending from a surface of the rotation channel; and
the lower coupling protrusion including a plurality of catches corresponding to the plurality of replaceable air treatment panels,
wherein one of the plurality of catches contacts the detent when the corresponding replaceable air treatment panel is in a maintenance access position.
